(19) United States
(12) Patent Application Publication
(10) Pub. No.: US 2023/0390184 A1
(43) Pub. Date: Dec. 7, 2023

(54) AND IMPROVED SKINCARE PRODUCTS

(71) Applicant: Sharon Skaar, Scottsdale, AZ (US)

(72) Inventor: Sharon Skaar, Scottsdale, AZ (US)

(21) Appl. No.: 16/952,139

(22) Filed: Nov. 19, 2020

(51) Int. Cl. A61K 8/9794; A61K 8/34; A61Q 19/10; A61K 8/9789; A61K 8/365; A61K 8/368

(52) U.S. Cl. CPC A61K 8/9794; A61K 8/347; A61Q 19/10; A61K 8/9789; A61K 8/365; A61K 8/368; A61K 2800/87; A61K 2800/28

(57) ABSTRACT

Described herein is a collection of skincare products include cleanser, weekly pad system, daily pad system, multi-purpose moisturizer, hydrating spritz, and eye cream. The products use scientifically proven active ingredients including retinol, hydroxy acids, Cannabidiol, and antioxidants to prevent skin aging. In further preferred embodiments, active agents including Vitamin C, peptides, HYASOL PF, algae extract are added.

### CROSS-REFERENCE

The present application claims priority to, U.S. provisional patent application No. 62/942,475, entitled “A UNIQUE FORMULATION OF SKINCARE PRODUCTS USING SCIENTIFICALLY PROVEN ACTIVE INGREDIENTS” filed Dec. 2, 2019, which is herein incorporated by reference in its entirety.

### BRIEF DESCRIPTION OF THE DRAWING

For a more complete understanding of the present invention, including its features and advantages, reference is now made to the detailed description of the invention taken in conjunction with the accompanying drawing in which:

FIG. 1 is a diagram utilized to explain the various embodiments of the present invention. An example of an individual 100 that would use the products, described herein, which are applied topically to the skin on the face 101, neck 102 and the chest 103. Applications have been shown to reduce the appearance of fine lines and wrinkles, reducing the affects of free radical damage and increasing dead skin cell turnover.

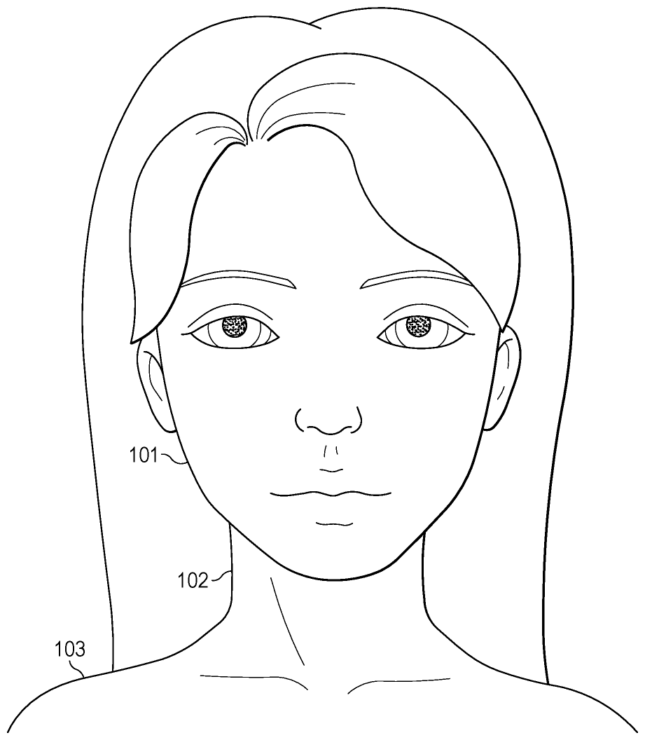

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

Skin aging is a complex biological process influenced by a combination of endogenous or intrinsic and exogenous or extrinsic factors. Because of the fact that skin health and beauty is considered one of the principal factors representing overall “well-being” in humans, several anti-aging strategies have been developed.

For cosmetologically strategies, active ingredients or active agents in a skincare product are the essential ingredients that cause physical changes in the skin. Current skin care products often contain many potentially irritating, allergenic, or harmful inactive ingredients. Therefore, a need exists for an effective formulation of skincare products using active agents to prevent aging.

The embodiments of the present invention are designed to solve many problems. For example, an improved, easy to use, and effective skincare collection in provided herein. The collection includes multi-purpose cleanser, weekly pad system, daily pad system, multi-purpose moisturizer, hydrating spritz, and eye cream. Several active ingredients are included in the products including retinol, hydroxy acids, and cannabidiol (CBD). Retinol is derived from Vitamin A and has been scientifically proven to build collagen. Hydroxy Acids are used for exfoliation of dead skin cells allowing new cell growth. CBD is used for medicinal or therapeutic purposes tided to the skin. These ingredients work by stimulating or impacting our skin's cannabinoid receptors to better regulate pain, inflammation, bacteria, lipid production (which can lead to acne), the release of histamine as well as assisting with skin cell proliferation.

In some embodiments, Vitamin C is also included in the form of BV-OSC. Its function is to assist in eliminating age spots, building collagen, and provide antioxidant protection.

In some embodiments, a minimum of 5% lactic acid is added to exfoliate the skin, lighten dark spots and improve the look of fine lines and wrinkles.

In some embodiments, bioactive peptides in the form of palmitoyl tripeptide, dipeptides, and SYN-TC are added to stimulate collagen synthesis in the skin.

In some embodiments, algae extract is added to draw toxins from the skin reducing the appearance of puffiness along with caffeine. Algae extract, along with detoxifying also softens, hydrates and conditions the skin.

In some embodiments, AcquaCell is added. AcquaCell is a natural complex combining watermelon rind extract, Lens esculenta fruit extract and apple extract in an optimized delivery system. Watermelon rind is one of nature's few materials to contain citrulline. Citrulline is essential to the functioning of filaggrin which forms a critical part of the skin's own water based moisturizing complex. The Lens esculenta fruit extract contains vitamin B5 and trisaccharides. The apple starch is a source of polysaccharides.

The collection of skincare products includes cleanser, weekly pad system, daily pad system, moisturizer, hydrating spritz, and eye cream. Three of the main active ingredients in the collection are retinol, hydroxy acids and CBD. Retinol is derived from Vitamin A and has been scientifically proven to build collagen. Hydroxy acids are used for exfoliation of dead skin cells allowing new cell growth. Cannabidiol CBD is used for medicinal or therapeutic purposes tied to the skin. It is believed to work by stimulating or impacting our skin's cannabinoid receptors to better regulate pain, inflammation, bacteria, lipid production, the release of histamine as well as assisting with skin cell proliferation.

### Multiple-Purpose Cleanser

The cleanser is a gel-based solution containing active ingredients including hydroxy acids and cannabidiol (CBD). Hydroxy acid is in the form of MFA complex which is a concentrated blend of Saccharium officinarurn (sugar cane) extract, Citrus limon (lemon) fruit extract, Citrus aurantium and Citrus aurantium dulcis (orange) peel oil. These extracts contain naturally occurring alpha hydroxy acids and antioxidants. Alpha hydroxy acids have been shown to promote smoother, younger looking skin by increasing the rate of cell renewal by means of exfoliation of surface layer dead cells. Antioxidants protect the new cells from damaging free radicals. CBD is an active ingredient in the cannabis plant. It is believed that it may reduce inflammation, bacteria, lipid production, the release of histamine as well as assisting with skin cell proliferation.

In certain embodiments, the concentration of MFA complex is a minimum of 0.5% for exfoliation, and CBD is at a minimum of 30 mg per ounce.

### Weekly Pad System

Pads are usually used in the application and the removal of makeup. Using a pad system to apply solution containing active ingredients once a week topically is beneficial for preventing skin aging. Here, the active ingredients in these pads are a minimum of 17% hydroxy acids, including alpha hydroxy, beta hydroxy and MFA complex. The breakdown of hydroxy acids is alpha hydroxy at a minimum of 7% in the form of glycolic acids, as alpha hydroxy acids have been shown to promote smoother, younger looking skin by increasing the rate of cell renewal by means of exfoliation of surface layer dead cells. Alpha hydroxy is derived from plants such as sugar cane, pineapple and sugar beets. Its botanical properties help to treat skin conditions such as psoriasis, melasma, and seborrheic keratosis. In this embodiment, the beta hydroxy acid is salicylic acid at a minimum of 2%. MFA complex is a concentrated blend of Saccharium officinarum (sugar cane) extract, Citrus limon (lemon) fruit extract, Citrus aurantium dulcis (orange) peel oil. These extracts contain naturally occurring alpha hydroxy acids and antioxidants. In this embodiment, the MFA complex is at a minimum of 3%.

Lactic acid can exfoliate the skin, lighten dark spots and improve the look of fine lines and wrinkles. In certain embodiments, a minimum of 5% lactic acid is used.

CBD may reduce inflammation, bacteria, lipid production, the release of histamine as well as assisting with skin cell proliferation. In one or more embodiments, cannabidiol CBD at a minimum of 30 mg per ounce is added to this powerful mix of hydroxy acids.

### Daily Pad System

A solution of specific active ingredients is applied topically by the use of a separate jar of disposable pads daily. One active ingredient in these pads include hydroxy acids in the form of MFA complex, retinol, and cannabidiol (CBD). MFA complex is a concentrated blend of Saccharium officinarum (sugar cane) extract, Citrus limon (lemon) fruit extract, Citrus aurantium dulcis (orange) fruit extract, Pyrus malus (apple) fruit extract, Camellia sinensis (green) tea extract, and Citrus aurantium dulcis (orange) peel oil. These extracts contain naturally occurring alpha hydroxy acids and antioxidants. Alpha hydroxy acids have been shown to promote smoother, younger looking skin by increasing the rate of cell renewal by means of exfoliation of surface layer dead cells. Antioxidants protect the new cells from damaging free radical. Retinol has been scientifically proven to boost the amount of collagen your body makes and plumps out skin, reducing fine lines and wrinkles. In this embodiment, Cannabidiol (CBD) reduces inflammation, bacteria, lipid production (which can lead to acne), the release of histamine as well as assisting with skin cell proliferation.

In certain embodiments, the concentration of MFA complex is a minimum of 3%; retinol is at a minimum of 0.025% deriving from vitamin A; CBD is added at a minimum of 30 mg per ounce.

In one or more embodiments, HYASOL PF is added at a minimum of 1%. HYASOL PF is an aqueous solution of high molecular mass hyaluronic acid for cosmetic application. The molecular mass of the hyaluronan polymer is about 1.6 mDa. It delivers outstanding tactile experience and skin revival immediately after application. It provides an excellent hydration capacity for perfectly moisturized skin and improves skin smoothness, elasticity and freshness.

In one or more embodiments, Vitamin C is also included in the form of BV-OSC. Its function is to assist in eliminating age spots, building collagen and provide antioxidant protection.

### Multi-Purpose Moisturizer

A cream-based formula is applied twice daily both morning and night after cleansing and using either the weekly pad or the daily pad. It contains powerful active ingredients including peptides, hydroxy acids, and cannabidiol (CBD). In this embodiment, Peptides in the form of palmitoyl tripeptide and dipeptides, SYN-TC are combined at a minimum of 2%. These specific peptides are highly bioactive, deeply skin penetrating peptides that are known to stimulate collagen synthesis in the skin. Collagens are the most abundant proteins in the skin, making up 75% of the skin proteins, and collagens are mainly responsible for the skin's strength and smoothness as they provide structure and firmness. However, over time collagen levels decrease and their proper function is reduced. SYN-TC is a synthetic peptide-based complex tailor-made for optimal activity. In vitro, SYN-TC peptides increase the formation of undamaged, stable and homogeneous collagens to facilitate their unique contribution to beautiful smooth looking skin. Hydroxy acids are in the form of MFA complex at a minimum of 2%. MFA complex is a concentrated blend of Saccharium officinarum (sugar cane) extract, Citrus limon (lemon) fruit extract, Citrus aurantium dulcis (orange) fruit extract, Pyrus malus (apple) fruit extract, Camellia sinensis (green) tea extract, and Citrus aurantium dulcis (orange) peel oil. These extracts contain naturally occurring alpha hydroxy acids and antioxidants. In this embodiment, CBD may reduce inflammation, bacteria, lipid production (which can lead to acne), the release of histamine as well as assist with skin cell proliferation. In addition, in this embodiment, CBD is added at a minimum of 30 mg per ounce.

In some embodiments, along with these primary active ingredients is the addition of acquaCell at a minimum of 2%. AcquaCell is a natural complex combining watermelon rind extract, Lens esculenta (lentil) fruit extract and apple extract in an optimized delivery system. Watermelon rind is one of nature's few materials to contain water based moisturizing complex. The lentil extract contains vitamin B5 and trisaccharide. The apple starch is a source of polysaccharides, sodium lactate and sodium PCA.

In certain embodiments, Vitamin C is also included in the form of BV-OSC. Its function is to assist in eliminating age spots, building collagen, and provide antioxidant protection.

### Hydrating Spritz

Hydrating spritz is a combination of coconut water, aloe leaf juice, and other active ingredients including AcquaCell, CBD, and HYASOL PF. AcquaCell is a natural complex combining watermelon rind extract, Lens esculenta (lentil) fruit extract and apple extract in an optimized delivery system. Watermelon rind is one of nature's few materials to contain water based moisturizing complex. The lentil extract contains vitamin B5 and trisaccharide. The apple starch is a source of polysaccharides, sodium lactate and sodium PCA. The apple starch is a source of polysaccharides, sodium lactate, and sodium PCA. CBD acts as a powerful antioxidant. HYASOL PF is added at a minimum of 1%. HYASOL PF delivers outstanding tactile experience and skin revival immediately after application. It provides an excellent hydration capacity for perfectly moisturized skin and improves skin smoothness, elasticity and freshness. HYASOL PF is an aqueous solution of high molecular mass hyaluronic acid for cosmetic application. The molecular mass of the hyaluronan polymer is about 1.6 mDa. In this embodiment, CBD is added at a minimum of 30 mg per ounce.

### Eye Cream

A rich cream-based formula is applied twice daily both morning and night after cleansing and using either the weekly pad or the daily pad and the moisturizer. It contains powerful active ingredients consisting of four different anti-aging peptides, bakuchiol, and HYASOL PF. The peptides are palmitoyl tripeptide-5, palmitoyl tripeptide-1, palmitoyl tetrapeptide-7, and dipeptide-1. These specific peptides are highly bioactive, deeply skin penetrating peptides that are known to stimulate collagen synthesis in the skin. Collagens are the most abundant proteins in the skin, making up 75% percent of the skin proteins. Collagens are mainly responsible for the skin's strength and smoothness as they provide structure and firmness. However, over time collagen levels decrease and their proper function is reduced. Bakuchiol is a plant-based retinol and CBD at a minimum of 30 mg per ounce. HYASOL PF is at a minimum of 1%. HYASOL PF delivers outstanding tactile experience and skin revival immediately after application. It provides an excellent hydration capacity for perfectly moisturized skin and improves skin smoothness, elasticity and freshness. HYASOL PF is an aqueous solution of highly molecular mass hyaluronic acid for cosmetic application. The molecular mass of the hyaluronan polymer is about 1.6 mDa.

In certain embodiments, the plant-based squalene and antioxidants are added to supply and support moisture balance around the delicate eye area. In certain embodiments, algae extract is added. Algae extract are powerful nutrients that draw toxins from the skin reducing the appearance of puffiness along with caffeine. Algae extract, along with detoxifying also softens, hydrates, and conditions the skin. Antioxidants in the form of green tea help fight the disgusting of aging caused by free radical damage.

1. A gel-based skincare cleansing solution comprising:
(a) hydroxy acids in the form of MFA Complex at a minimum of 0.5% wherein MFA complex comprises concentrated blend of Saccharium officinarum extract, Citrus limon fruit extract, Citrus aurantium dulcis fruit extract, Pyrus malus fruit extract, Camellia sinensis tea extract, and Citrus aurantium dulcis peel oil; and
(b) a minimum of 1 mg cannabidiol (CBD) per ounce.

2. A weekly pad system for applying a solution once a week comprising:
a minimum of 17% hydroxy acids including alpha hydroxy, beta hydroxy, and MFA complex wherein (i) alpha hydroxy is at a minimum of 7% in the form of glycolic acid; (ii) beta hydroxy acid is salicylic acid at a minimum of 2%; and (iii) MFA is a concentrated blend at a minimum of 3% comprising Saccharium officinarum extract, Citrus limon fruit extract, Citrus aurantium dulcis fruit extract, Pyrus malus fruit extract, Camellia sinensis tea extract, and Citrus aurantium dulcis peel oil.

4. The weekly pad system of claim 4 wherein cannabidiol (CBD) is added at a minimum of 30 mg per ounce.

5-10. (canceled)
